(19) United States
(12) Patent Application Publication
(10) Pub. No.: US 2002/0068314 A1
(43) Pub. Date: Jun. 6, 2002

(54) Method of detecting mycobacteria

(76) Inventor: Michael H. Levi, Brooklyn, NY (US)

Correspondence Address: James V. Costigan, Esq., HEDMAN & COSTIGAN, P.C., Suite 2003, 1185 Avenue of the Americas, New York, NY, 10036-2646, US

(21) Appl. No.: 09/885,304

(22) Filed: Jun. 20, 2001

Related U.S. Application Data
(60) Provisional application No. 60/213,810, filed on Jun. 22, 2000.

(51) Int. Cl. C12Q 1/04; C12N 1/20

(52) U.S. Cl. 435/34; 435/253.1; 435/253.6

(57) ABSTRACT

The present invention describes a method of growth enhancement of M. tuberculosis and other mycobacteria species by using natural or synthetic hemoglobin as a supplement to conventional media that is formulated to support growth of mycobacterial organisms.

This application claims the benefit of Provisional Application Serial No. 60/213,810 filed Jun. 22, 2000.

### BACKGROUND OF THE INVENTION

M. tuberculosis has a generation time of 18 to 22 hours and cultures require a very long period of incubation before growth of the organism can be observed. Rapid detection of M. tuberculosis in clinical specimens is a crucial goal in the art of commercial bacteriologic media formulation. The period between obtaining a culture from a patient and the detection of mycobacterial growth represents a time delay in confirmation of diagnosis that could be shortened considerably by a media supplement that increases the growth rate of the organism. With shortened recovery time, antibiotic sensitivity studies can also be commenced earlier, thereby enabling clinicians to adjust therapy so that the patient receives the most appropriate treatment based on the results of the antibiotic profile of the isolate. The invention thus confers both public health benefits and benefits to the treated individuals, by aiding the clinician in obtaining a faster diagnosis, and effecting an earlier assessment of the adequacy of therapy regimens.

Using traditional media, mycobacterial species can be commonly detected within 3 to 6 weeks. State of the art media such as that sold by Becton Dickinson detects mycobacteria over several hours or days. The present invention shortens the period to detection of mycobacterial growth by about 25%.

Kondo et al. (Kekkaku: 67:97-105) discloses supplementing media such as Middlebrook 7H9, Dubos, Ogawa, or Kudo PD with culture filtrates of Gemella hemolysans. The filtrate was added to the media along with whole eggs and brain heart infusion and 7% human blood to promote growth of the target organisms. Only the use of the filtrate of Gemella hemolysans is taught by Kondo et al.

### SUMMARY OF THE INVENTION

The present invention provides an improved bacteriological media for growing suspected cultures of mycobacteria. The media is a conventional media for growing mycobacteria to which has been added a quantity of natural or synthetic hemoglobin which is effective to increase the growth rate of a mycobacteria.

Accordingly, it is a primary objective of the invention to provide a novel method for supporting the growth of mycobacteria which provides for faster growth of the mycobacteria.

It is also an object of this invention to provide an improved bacteriological media which accelerates the growth of mycobacteria.

It is also an object of this invention to provide a method for reducing the detection time for detecting a mycobacteria which avoids the need to culture a Gemella hemolysans organism and recover the filtrate from such a culture.

### DETAILED DESCRIPTION OF THE INVENTION

The invention describes a method of supplementation of commercial mycobacterial culture media with a natural or synthetic hemoglobin source in combination with a conventional media which supports the growth of mycobacteria. This invention is primarily concerned with the culture of Mycobacterium tuberculosis from sputum samples. The addition of the hemoglobin source to the conventional media results in a shortened recovery time for the organism and therefore enhanced turn-around time to final clinical diagnosis. Generally from 1 mg to 30 mg of hemoglobin is added per ml of culture medium. Good results have been obtained with 10 mg of hemoglobin/ml of culture medium. It has been demonstrated that 2 mg/ml of a hemoglobin additive (Sigma) reduces the time to detection of a mycobacteria sample by 1.5 days and the use of 10 mg/ml of the same hemoglobin reduces the time for detection by 2.8 days. Natural human hemoglobin is commercially available and synthetic hemoglobins are also described in the literature as hemoglobin which has one or more modifications to the structure of hemoglobin which do not modify the activity of the hemoglobin molecule.

An example of a media which supports the growth of mycobacteria is a glycerin based media having a protein source such as egg protein or casein which is adjusted to a pH of 7.4-8.0. Other media is described in Kondo et al. A preferred media is commercially available as Bactec media from Becton Dickinson and Company which has the following composition:

| | |
|---|---|
| purified water | 40 ml |
| 7H9 Middlebrook Broth base | 0.47% w/v |
| casein hydrolysate | 0.10% w/v |
| Supplement H | 0.3% w/v |
| glycerin | 0.10% w/v |
| ammonium sulfate | 0.05% w/v |
| ferric ammonium citrate | 0.006% w/v |
| polysorbate 80 | 0.0025% w/v |
| hemin | 0.0005% w/v |

This media requires the addition of 2.0 ml of Panta F solution prior to use.

### DESCRIPTION OF A PREFERRED EMBODIMENT

A series of experiment was carried out to compare the number of hours of culture growth to reach detection of a mycobacteria when a source of hemoglobin was added to a mycobacteria culture. The media that was used was the Bactec media with Panta F with or without a source of human hemoglobin using a Bactec 9000 as the detecting means where a decrease in oxygen increases fluorescence.

Two experiments were carried out using a filtrate of a Gemella hemolysans culture which contained 7% human blood and unmodified Bactec media with Panta F as controls and experiments were carried out using hemoglobin from Sigma, hemoglobin obtained from a lysate of whole blood and ovine hemoglobin from Difco. The results were as shown in Table 1:

TABLE 1

| Hb Source | | | | |
|---|---|---|---|---|
| Ovine | Control | Gemella Lys. | Human_{Sigma} | Human_{lysate} |
| Hours to | 233 (33) | 161 (0.6) | 166 (2) | 176 (14) |
| Detect. 204 () | | | | |
| Hb Conc. | 0 | 7 10 | 4 | 10 |
| (mg/ml) | | | | |

The Gemella hemolysans preparation of human blood was fractionated and the fraction having the greatest effect on time to detection was the molecular weight fraction above 30,000 as obtained using a 30 kD filter.

Tests carried out with another strain of M. tuberculosis have resulted in an even greater reduction in detection time (41 vs. 80 hours) which has confirmed the validity of the test.

1. A method for increasing the growth rate of a mycobacteria species which comprises growing said mycobacteria in a culture media which contains natural or synthetic human hemoglobin.

2. A method as defined in claim 1 wherein the mycobacteria is Mycobacterium tuberculosis.

3. In a media for growing mycobacteria, the improvement which comprises adding an amount of natural or synthetic human hemoglobin which increases the growth rate of said mycobacteria.

4. A media as defined in claim 3 which contains from 1 mg/ml to 30 mg/ml of hemoglobin.

5. A method for the determination of mycobacteria which comprises culturing an unknown sample in a media which comprises a natural or synthetic hemoglobin which increases the growth rate of a mycobacteria.
